(19) United States
(12) Patent Application Publication
(10) Pub. No.: US 2017/0164880 A1
(43) Pub. Date: Jun. 15, 2017

(54) OBJECT INFORMATION OBTAINING APPARATUS AND OBJECT INFORMATION OBTAINING METHOD

(71) Applicant: CANON KABUSHIKI KAISHA, Tokyo (JP)

(72) Inventor: Kohtaro Umezawa, Tokyo (JP)

(21) Appl. No.: 15/374,744

(22) Filed: Dec. 9, 2016

(30) Foreign Application Priority Data
Dec. 9, 2015 (JP) 2015-240582

(51) Int. Cl. A61B 5/1455; A61B 5/00

(52) U.S. Cl. CPC A61B 5/14552; A61B 5/0095

(57) ABSTRACT

An object information obtaining apparatus includes a conversion element configured to receive a photoacoustic wave generated in an object in response to irradiation with light from a light irradiation unit and convert the received photoacoustic wave into an electric signal, a light quantity distribution obtaining unit configured to obtain light quantity distribution information in the object, a first distribution obtaining unit configured to obtain distribution information of a photoacoustic wave generation source in the object, a correction unit configured to correct the distribution information and also correct the light quantity distribution information in correspondence with the correction, and a second distribution obtaining unit configured to obtain characteristic distribution information in the object by using the corrected distribution information and the corrected light quantity distribution information.

### BACKGROUND

1. Field of the Disclosure

The present disclosure relates to an object information obtaining apparatus that obtains information about an object, and a method for obtaining information about an object.

2. Description of the Related Art

In the medical field, studies retarding imaging of a biologic object are ongoing, and one of them is photoacoustic imaging (PAI).

In the photoacoustic imaging, an object is irradiated with pulsed light and absorbs energy of this irradiation light, thereby generating an acoustic wave (hereinafter referred to as a photoacoustic wave). Then, this photoacoustic wave is received by a conversion element such as a piezoelectric element, and a received signal therefrom is subjected to analysis processing in a processor, by which a distribution regarding an optical characteristic value inside the object is obtained as image data.

Examples of the distribution regarding the optical characteristic value include a distribution of a sound pressure generated in response to optical absorption (an initial sound pressure distribution) and a distribution of an optical absorption coefficient. Further, a concentration-related distribution of a substance existing in the object (a distribution of values regarding concentration of the substance) can also be obtained as the distribution regarding the optical characteristic value, by irradiating the object with pulsed light of mutually different wavelengths between measurements on a same position to obtain the optical absorption coefficient for corresponding wavelength, and making a comparative calculation between the absorption coefficients, each of which is obtained for the corresponding wavelength.

Examples of the concentration-related distribution include a distribution of a content rate of oxyhemoglobin with respect to a total amount of hemoglobin in blood, i.e., an oxygen saturation distribution in the blood.

The oxygen saturation distribution can be obtained by comparing measurement results obtained by irradiating the object with light of mutually different wavelengths between measurements on a same position, e.g., an identical blood vessel. Accordingly, if the object moves when switching the wavelength, this movement leads to inconsistency in a positional relationship of the measured sites (for example, the same blood vessel), resulting in occurrence of an error in a calculated value of the oxygen saturation distribution. As one measure against this problem, Japanese Patent Application Laid Open No. 2011-177496 discusses a technique for reducing the error in the value of the oxygen saturation distribution due to the movement of the object. FIGS. 10A, 10B, and 10C illustrate the technique discussed in Japanese Patent Application Laid-Open No. 2011-177496. FIG. 10A illustrates that an absorption coefficient distribution A obtained from irradiation with light having a first wavelength, and an absorption coefficient distribution B obtained irradiation with light having a second wavelength are measured with a positional deviation occurring between them due to the movement of the object. More specifically, characteristic portions (a light circle in the absorption coefficient distribution A and a dark circle in the absorption coefficient distribution B) such as blood vessels that should be located at the same position deviate from each other in FIG. 10A. Then, as illustrated in FIG. 10B, a spatial resolution of the absorption coefficient distribution data B is adjusted so as to fall below a spatial resolution of the absorption coefficient distribution data A, thereby allowing the characteristic portion in the absorption coefficient distribution data A to be encompassed in the characteristic portion in the absorption coefficient distribution data B. This adjustment allows the same characteristic portions (the blood vessels) to be used in the calculation at the time of the comparative calculation between these pieces of absorption coefficient distribution data A and B, thereby reducing the influence of the positional deviation (FIG. 10C).

The technique discussed in Japanese Patent Application Laid-Open No. 2011-177496 lowers the spatial resolution of one of the pieces of distribution data (the absorption coefficient distribution data B), i.e., blurs the distribution data. Accordingly, the oxygen saturation can be relatively accurately calculated regarding an area where the characteristic portion the absorption coefficient distribution data A (the light circle in the drawing) and the characteristic portion in the absorption coefficient distribution data B (the dark circle in the drawing) overlap each other in FIG. 10B, but is not appropriately quantitated regarding an area where they do not overlap each other. More specifically, an area in the characteristic portion in the absorption coefficient distribution data B illustrated in FIG. 10B that does not overlap the characteristic portion in the absorption coefficient distribution data A is not actually the characteristic portion such as the blood vessel but is nevertheless treated as the characteristic portion, thereby leading to error in the result of the comparative calculation. Therefore, the technique discussed in Japanese Patent Application Laid-Open No. 2011-177496 has been facing a demand for further improvement in terms of accuracy of the calculation of the distribution information.

### SUMMARY

The present disclosure is directed to improving the accuracy of the calculation of the distribution information regarding the optical characteristic value in the object.

According to one or more aspects of the present disclosure, an object information obtaining apparatus includes a light irradiation unit, a conversion element configured to receive a photoacoustic wave generated in an object in response to irradiation with light from the light irradiation unit and convert the received photoacoustic wave into an electric signal, a light quantity distribution obtaining unit configured to obtain light quantity distribution information representing a distribution state of a light quantity in the object with respect to the light with which the object is irradiated from the light irradiation unit, a first distribution obtaining unit configured to obtain distribution information representing a distribution state of a photoacoustic wave generation source in the object by using the electric signal, a correction unit configured to correct the distribution information obtained by the first distribution obtaining unit and also correct the light quantity distribution information obtained by the light quantity distribution obtaining unit in correspondence with the correction, and a second distribution obtaining unit configured to obtain characteristic distribution information in the object by using the corrected distribution information and the corrected light quantity distribution information.

According to another aspect of the present disclosure, an object information obtaining method includes obtaining light quantity distribution information representing a distribution state of a light quantity in an object with respect to light with which the object is irradiated, obtaining distribution information representing a distribution state of a photoacoustic wave generation source in the object by using an electric signal obtained by receiving a photoacoustic wave generated from the object irradiated with the light, correcting the obtained distribution information and also correcting the obtained light, quantity distribution information in correspondence with the correction, and obtaining characteristic distribution information. In the object by using the corrected distribution information and the corrected light quantity distribution information.

Further features of the present disclosure will become apparent from the following description of exemplary embodiments with reference to the attached drawings.

### BRIEF DESCRIPTION OF THE DRAWINGS

FIG. 1 is a schematic diagram illustrating a configuration of a photoacoustic apparatus according to one or more aspects of the present disclosure.

FIG. 2 is a flowchart illustrating an operation of a signal processing unit according to one or more aspects of the present disclosure.

FIG. 3 is a schematic diagram illustrating the operation of the signal processing unit according to one or more aspects of the present disclosure.

FIG. 4 is a schematic diagram illustrating an example of a configuration of the signal processing unit according to one or more aspects of the present disclosure.

FIGS. 5A, 5B, 5C, 5D, 5E, and 5F are schematic diagrams illustrating the operation of the signal processing unit according to one or more aspects of the present disclosure.

FIG. 6 is a schematic diagram illustrating a configuration of a photoacoustic apparatus according to one or more aspects of the present disclosure.

FIG. 7 is a schematic diagram illustrating one example of a display screen according to one or more aspects of the present disclosure.

FIG. 8 is a flowchart illustrating an operation of a signal processing unit according to one or more aspects of the present disclosure.

FIG. 9 is a flowchart illustrating an operation of a signal processing unit according to one or more aspects of the present disclosure.

FIGS. 10A, 10B, and 10C are schematic diagrams illustrating a conventional technique.

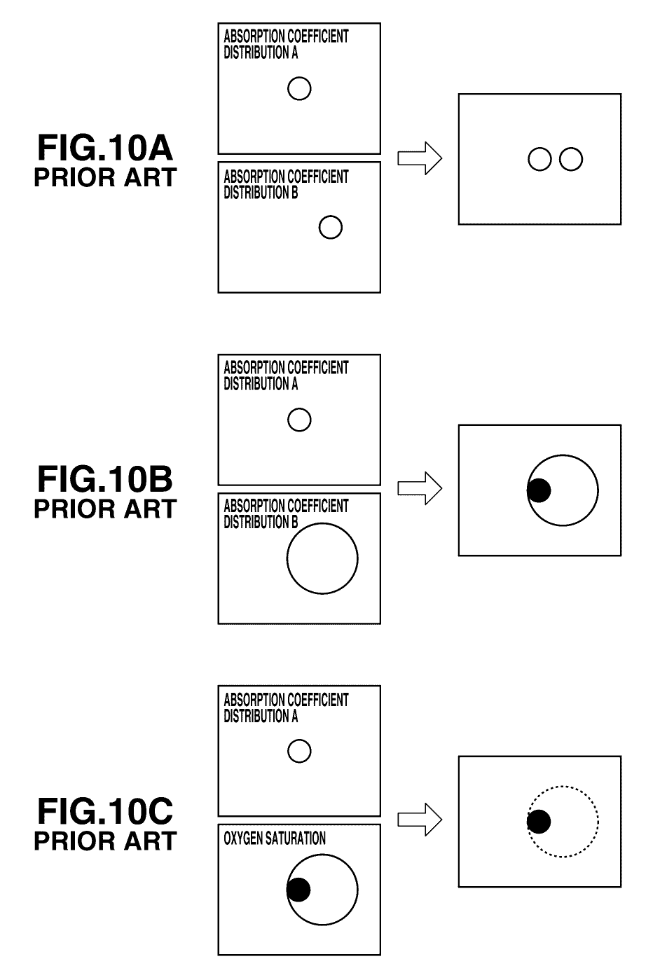

### DESCRIPTION OF THE EMBODIMENTS

The present disclosure with respect to obtaining of information regarding a characteristic in an object with use of a photoacoustic signal generated from irradiation or the object with light be described according to one or more aspects of the present disclosure.

In the following description, of the exemplary embodiments of the present disclosure will be described with reference to the drawings. First, an overview of a technique employed in the exemplary embodiments of the present disclosure will be described, and, after that, the individual exemplary embodiments will be described. Further, in the following description, like components will be identified by like once numerals in principle, and redundant descriptions will not be repeated.

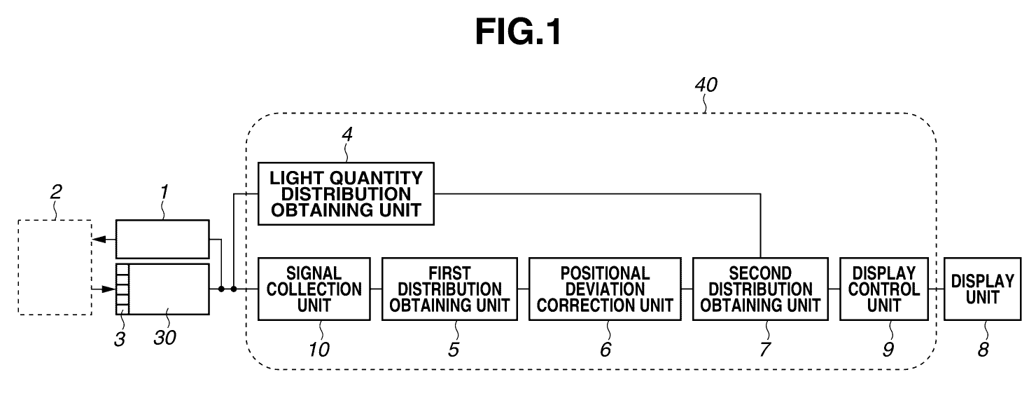

As illustrated in FIG. 1, an object information obtaining apparatus according to one or more aspects of the present disclosure includes a light; irradiation unit 1, and a conversion element(s) 3, each of which receives a photoacoustic wave generated in an object 2 in response to irradiation with light from the light irradiation unit 1 and converts the received photoacoustic wave into an electric signal. The object information obtaining apparatus further includes a light quantity distribution obtaining unit 4, which obtains light quantity distribution information representing a distribution state of a light quantity in the object 2 with respect to the light with which the object 2 is irradiated by the light irradiation unit 1, and a first distribution obtaining unit 5, which obtains distribution information representing a distribution state of a photoacoustic wave generation source in the object 2 with use of the electric signal output from each of the conversion elements 3. The object information obtaining apparatus further includes a positional deviation correction unit 6, which is a correction unit that corrects the distribution information obtained by the first distribution obtaining unit 5 and also corrects the light quantity distribution information obtained by the light quantity distribution obtaining unit 4 in correspondence with this correction. Then, the object information obtaining apparatus includes a second distribution obtaining unit 7, which obtains characteristic distribution information in the object 2 with use of the corrected distribution information and the corrected light quantity distribution information. With this configuration, the object information obtaining apparatus can improve the accuracy of the calculation of the distribution information regarding the optical characteristic value in the object 2. Details thereof will be described below. In the followings, a description is given of a case where the object information obtaining apparatus accurately obtains the oxygen saturation distribution information by way of example, but this is a technique applicable to not only the oxygen saturation distribution information but also the absorption coefficient distribution information and the like.

The oxygen saturation distribution information represents how an oxygen saturation value is distributed at each point (position), and thus the oxygen saturation should be accurately obtained at each point to improve the accuracy when the oxygen saturation distribution information is obtained. As described above, since the oxygen saturation is obtained by making the comparative calculation between the absorption coefficient values obtained by irradiating the object with the light of mutually different wavelengths between measurements on a same position, the absorption coefficient should be correctly calculated at each point to improve the accuracy in calculating the oxygen saturation at each point. Then, when a ratio between the calculated absorption coefficients is calculated, it is extremely important to calculate a ratio of the absorption coefficients at the same point, i.e., the absorption coefficients at the same site of the object 2 (for example, the absorption coefficients at the same blood vessel (the absorption coefficients at blood in the same blood vessel)).

However, as described above, for example, when the wavelength is switched, the positional deviation (a change in a positional relationship between the object 2 and the conversion element 3) may occur due to a movement of the object body (pulsation, breathing, or the like).

Accordingly, the oxygen saturation distribution information can be obtained with improved accuracy by carrying out a correction for resolving the positional deviation, in particular, carrying out registration between the two pieces of absorption coefficient distribution information, and calculating the ratio between the pieces of absorption coefficient distribution information registered to each other.

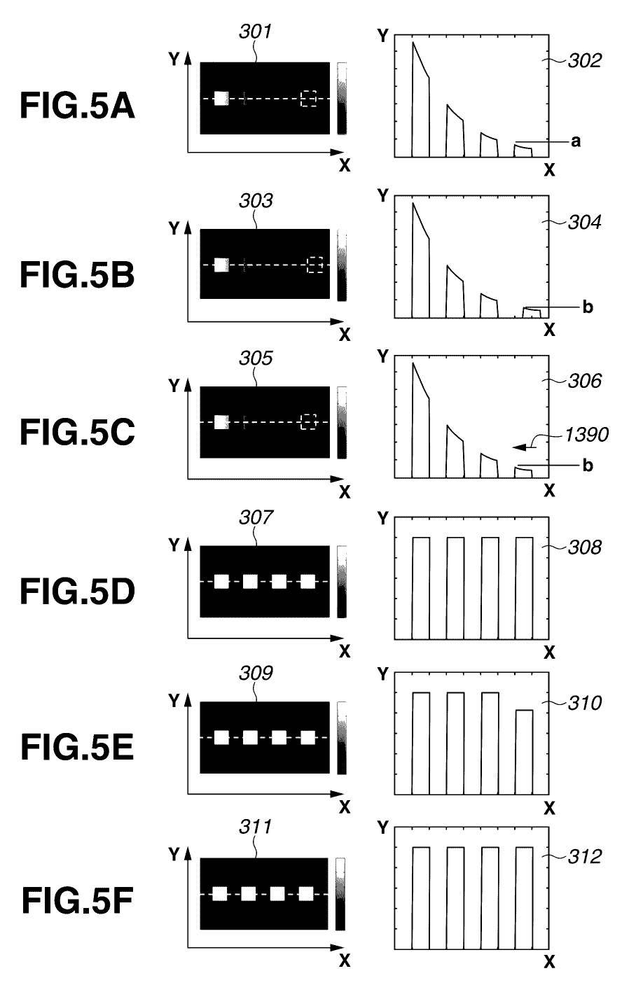

As will be described below in detail the absorption coefficient distribution information is a value calculated by dividing the initial sound pressure distribution information which the information about the photoacoustic wave generation source distribution that is obtained by receiving the photoacoustic wave generated from the irradiation of the object 2 with the light, by the light quantity distribution information, which the information about the distribution of the light quantity in the object 2. Accordingly, the correction of the positional deviation (registration) between the two pieces of absorption coefficient distribution information obtained by irradiating the object with the light of mutually different wavelengths between measurements on a same position can be achieved by a correction of a positional deviation between the two pieces of initial sound pressure distribution information obtained by irradiating the object with the light of mutually different wavelengths between measurements on a same position. However, it has been revealed from a result of our diligent intense study that it is insufficient to just correct the positional deviation between the two pieces of initial sound pressure distribution information. Details thereof will be described now with reference to FIGS. 5A, 5B, 5C, 5D, 5E, and 5F. A Y axis and an X axis in left illustrations of FIGS. 5A to 5F represent a position on an XY plane, and an X axis in right illustrations of FIGS. 5A to 5F represents a position in an X axis direction of the XY plane. Further, a Y axis in the right illustration represents a sound pressure intensity in FIGS. 5A to 5C, and an absorption coefficient value in FIGS. 5D to 5F.

FIGS. 5A, F, 50, 5D, 5E, and 5F are each a diagram illustrating the initial sound pressure distribution information and the absorption coefficient distribution information obtained by irradiating a phantom, in which targets (photoacoustic wave generation sources) imitating four blood vessels having a same absorption coefficient were arranged at intervals in the X direction, with the light from a left side of the diagram. FIG. 5A illustrates an initial sound pressure distribution 301 obtained by irradiating the phantom with the light at time t0, and a sound pressure intensity (a brightness in an image) 302 at each of points located on a white dotted line illustrated in FIG. 5A. Further, FIG. 5B illustrates an initial sound pressure distribution 303 obtained by irradiating the phantom with the light at time t1 different from the time t0 (for example, a time after the wavelength was switched), and a sound pressure intensity (a brightness in an image) 304 at each of points located on a white dotted line illustrated in FIG. 5B.

Further, FIG. 5C illustrates a corrected initial sound pressure distribution 305 obtained by carrying out a registration correction (positional deviation correction) on the initial sound pressure distribution 303 at time t1 so as to register it to initial sound pressure distribution 301 at time t0, and a sound pressure intensity 306 thereof. In each of FIGS. 5A, 5B, and 5C, a rightmost target is hard to see due to its low intensity, and is indicated while being surrounded by a white dotted square in each of the initial sound pressure distributions 301, 303, and 305 for the sake of convenience. Then, the position of the rightmost target in the drawings deviated at time t1 (FIG. 5B) relative to time t0 (FIG. 5A), so that it can be confirmed that the position of the rightmost target deviated and the sound pressure intensity thereof also reduced from a value a to a value b in the initial sound pressure distribution 303 and the sound pressure intensity 304 illustrated in FIG. 5B. More specifically, the rightmost target shifted to the right, which prohibited a sufficient light quantity from reaching this target, resulting in the reduction in the initial sound pressure value.

Further, FIG. 5D illustrates absorption coefficient distribution information 307 obtained by making the calculation on the initial sound pressure distribution 301 with use of (in particular, by dividing the initial sound pressure distribution 301 by) the light quantity distribution information about the light with which the phantom was irradiated at time t0, and an absorption coefficient value (an intensity) 308 at each of points on a white dotted line. Further, FIG. 5E illustrates absorption coefficient distribution information 309 obtained by making the calculation on the corrected initial sound pressure distribution 305 after the registration correction carried out on the initial sound pressure distribution 303 at time t1, with use of the light quantity distribution information about the light with which the phantom was irradiated at time t1 without any correction carried out thereon, and an absorption coefficient value (an intensity) 310 at each of points on a white dotted line. FIG. 5E illustrates an example that did not employ the exemplary embodiments of present disclosure. On the other hand, FIG. 5F illustrates an example that employed the exemplary embodiments of the present disclosure. More specifically, FIG. 5F illustrates absorption coefficient distribution information 311 obtained by making the calculation on the above-described corrected initial sound pressure distribution 305 with use of the light quantity distribution information about the light with which the phantom was irradiated at time t1 after a correction carried out thereon in correspondence with this registration correction, and an absorption coefficient value (an intensity) 312 at each of points on a white dotted line. As understood from the illustration of the drawings, it can be confirmed that the rightmost absorption coefficient in the drawing yielded a lower value than the other three coefficients in the absorption coefficient distribution information 309 and the absorption coefficient value 310 illustrated in FIG. 5E, while all the four absorption coefficients were able to yield a same value in FIG. 5F.

In sum, correcting the positional deviation between the two pieces of initial sound is pressure distribution information obtained by, for example, irradiating the object with light of mutually different wavelengths between measurements on a same position improves the result in such a manner that the pieces of data obtained from the same point of the object 2, such as the same blood vessel, are located at the same position (refer to the initial sound pressure distribution 305 illustrated in FIG. 5C). Then, this correction should allow the pieces of data obtained from the same point to be used in the calculation (for example, the calculation of the ratio between the absorption coefficients at the same blood vessel), but, actually, is insufficient in terms of the accuracy of the calculation of the absorption coefficient at this point as illustrated in FIG. 5E. More specifically, the correction of the positional deviation, unless being also carried out on the light quantity distribution information in correspondence with this correction of the positional deviation on the initial sound pressure distribution information, results in a conversion of the initial sound pressure information into the absorption coefficient information with use of a different light quantity from the light quantity actually delivered to the blood vessel, and therefore does not reach a correct calculation of the above-described absorption coefficient at each of the points. Referring to the specific example, in the initial sound pressure distribution information 305, the rightmost target was subjected to the correction of the positional deviation and was displaced to the left (an arrow 1390) from ins original position. However, the rightmost target was actually located on a further right side, i.e., on a further deep side of the object so that the light reached this target only by, for example, I₁. Nevertheless, the initial sound pressure thereof was divided by a light quantity (I₂ (>I₁)) delivered to a shifted to the left from its original position, and therefore was converted into a smaller value than the actual value as the absorption coefficient (refer to FIG. 5E). A failure to obtain the correct absorption coefficient distribution information in this manner also makes it impossible to obtain the oxygen saturation distribution information, which is derived by calculating the ratio therebetween, correct distribution information.

To that end, on the basis of this novel discovery, the exemplary embodiments according to the present disclosure correct the initial sound pressure distribution information (the distribution information indicating the distribution state of the position of the photoacoustic wave generation source), and also correct the light quantity distribution information in correspondence with this correction. More specifically, the exemplary embodiments according to the present disclosure correct the light quantity distribution information in such a manner that a relationship between the distribution state of the photoacoustic wave generation source (the target) obtained by the first distribution obtaining unit 5 and the distribution state of the light quantity obtained by the light quantity distribution obtaining unit 4, and a relationship between the corrected distribution state of the photoacoustic wave generation source and the corrected distribution state of the light quantity match each other. Then, the exemplary embodiments according to the present disclosure obtain the characteristic information such as the absorption coefficient distribution information and the oxygen saturation distribution information with use of the corrected initial sound pressure distribution information and the corrected light quantity distribution information, thereby improving the accuracy of obtained information (refer to FIG. 5F).

Now, each of the exemplary embodiments of the present disclosure will be described with reference to the drawings. The present disclosure is not limited to only the object information obtaining apparatus represented by a photoacoustic apparatus, but also covers method for obtaining the object information and a program for performing this method.

Now, a configuration and processing of the object information obtaining apparatus according to a first exemplary embodiment will be described.

### (Overall Configuration of Apparatus)

FIG. 1 is a schematic diagram illustrating a configuration of a photoacoustic apparatus, which is the object information obtaining apparatus according to one or more aspects of the present disclosure. The photoacoustic apparatus according to the present exemplary embodiment includes at least the light irradiation unit 1, a probe 30 including the conversion elements 3, each of which receives the photoacoustic wave generated in the object 2 in response to the irradiation with the light from the light irradiation unit 1 and converts the received photoacoustic wave into the electric signal, and a signal processing unit 40, which performs signal processing with use of a reception signal output from each of the conversion elements 3. The signal processing unit 40 includes at least the above-described light quantity distribution obtaining unit 4, the above-described first distribution obtaining unit the above-described positional deviation correction unit 6, and the above-described second distribution obtaining unit 7. Further, as a desirable configuration, the signal processing unit 40 can also include a display control unit 9 and a signal collection unit 10, which will be described below, as illustrated in FIG. 1.

The object 2 is irradiated with the light output from the light irradiation unit 1 via an optical propagation member (not illustrated), such as a fiber and a lens. The object 2 is irradiated a plurality of times with the pulsed light at different timings. The light with which the object 2 is irradiated is propagated and diffused in the object 2, and is absorbed by a substance existing in object 2. Such an optically absorbing substance absorbs energy of the pulsed light and generates a photoacoustic wave each time the pulsed light is emitted. More specifically, a first photoacoustic wave is generated in response to irradiation with light at a first time, and a second photoacoustic wave is generated in response to irradiation with light at a second time. The generated photoacoustic waves are propagated in the object 2 and reach the conversion elements 3. The conversion elements 3 are provided so as to achieve photoacoustic matching with the object 2.

Each of the plurality of conversion elements 3 outputs the reception signal, which is a chronological electric signal, in response to receiving the photoacoustic wave. More specifically, each of the conversion elements 3 outputs a chronological first reception signal in response to receiving the first photoacoustic wave, and outputs a chronological second reception signal in response to receiving the second photoacoustic wave. The output reception signals are input to the signal processing unit 40. The reception signal is sequentially input to the signal processing unit 40 each time the object 2 is irradiated with the pulsed light. The signal processing unit 40 generates distribution such as the characteristic distribution and the concentration-related distribution based on the optical absorption in the object 2 with use of the input reception signal. Further, the signal processing unit 40 generates image data based on the generated distribution, and displays an image on a display unit 8.

In a case where the photoacoustic apparatus is an apparatus designed to examine a relatively small object as an examination target, such as a photoacoustic microscope, the probe 30 may include only one conversion element 3. However, it is desirable that the probe 30 includes the plurality of conversion elements in a case where the photoacoustic apparatus is an apparatus designed to examine a relatively large object, such as a breast, as the examination target.

### (Internal Configuration of Signal Processing Unit 40)

Next, a configuration of the signal processing unit 40 according to the present exemplary embodiment will be described.

The signal collection unit 10 collects the chronological analog reception signal output from each of the plurality of conversion elements 3 for each channel, and performs signal processing such as amplification of the reception signal, an analog-to-digital (AD) conversion of the analog reception signal, and a storage of the digitalized reception signal.

Desirably, the light quantity distribution obtaining unit 4 obtains a light quantity distribution φ by making a calculation from the distribution of the light with which the object 2 is irradiated in consideration of light propagation inside the object 2. At this time, the light quantity distribution obtaining unit 4 may make the calculation in consideration of a shape of the object 2. More specifically, the light quantity distribution obtaining unit 4 calculates a background optical coefficient in the object 2 by measuring the light propagated in the object 2 with use of a light quantity meter (e.g., Time Resolved Spectroscopy (TRS) 20 manufactured by Hamamatsu Photonics K.K.), and calculates the light quantity distribution information inside the object 2 with use of an optical diffusion equation based on the calculated background optical coefficient, information indicating the outer shape of the object 2, and information indicating the distribution of the laser irradiation light.

The first distribution obtaining unit 5 obtains the initial sound pressure distribution with use of the reception signal output from the signal collection unit 10 in the present exemplary example, and obtains a plurality of pieces of distribution information, each of which is obtained for the corresponding irradiation with the light from the light irradiation unit 1. An absorption coefficient μₐ at a certain position (i, j, k) in the object 2 can be obtained with use of an expression 1. Assume that i, j, and k are each an integer indicating a coordinate in the object 2.

P=Γ·μₐ·φ Expression 1

In the expression 1, P, Γ, and φ represent an initial sound pressure (a generated sound pressure) at the position (i, j, k), a Gruneisen constant, and a light quantity delivered to he position (i, j, k), respectively.

The initial sound pressure P at the position (i, j, k) on three-dimensional space coordinates can be obtained by reconstructing an image after applying a filter for correcting a band of the probe 3 based on the reception sign for each channel that is output from the signal collection unit 10. For example, a known reconstruction method such as the universal back-protection (UBP) and the filtered back-projection (FBP) discussed in U.S. Pat. No. 5,713,356 can be employed as the image reconstruction method. Alternatively, phasing and summing (Delay and Sum) processing may be employed as the image reconstruction method.

The first distribution obtaining unit 5 can obtain the initial sound pressure at each point (position) by performing this image reconstruction processing with respect to each position, and therefore can obtain the initial sound pressure distribution. The initial sound pressure distribution may be three-dimensional distribution data (data formed from a collection of voxels) corresponding to a certain region in the object 2, or may be two-dimensional distribution data (data formed from a collection of pixels) corresponding to one cross section therein. In this manner, the first distribution obtaining unit 5 obtains the plurality of pieces of distribution information, each of which is obtained for the corresponding irradiation with the light from the light irradiation unit 1.

In a case where the photoacoustic apparatus is an optical focusing photoacoustic microscope or an acoustic focusing photoacoustic microscope using a focusing probe, the first distribution obtaining unit 5 can also generate the distribution data without performing the image reconstruction processing. More specifically, the probe 30 and a light irradiation spot are moved relative to the object 2 by a scanning mechanism (not illustrated), and the probe 30 receives the photoacoustic waves at a plurality of scanning positions. Then, after the obtained reception signals with respect to a change over time has been subjected to envelope detection, the first distribution obtaining unit 5 converts a temporal axis direction of the signal for each of the light pulses into a depth direction of the object 2, and plots the conversion result on the spatial coordinates. The first distribution obtaining unit can construct the distribution data by performing this processing for each of the scanning positions. In this manner, the first distribution obtaining unit 5 according to the present exemplary embodiment obtains initial sound pressure distribution each time the object 2 is irradiated with pulsed light emitted from the light irradiation unit 1, and outputs the obtained initial sound pressure distribution to the positional deviation correction unit 6.

The positional deviation correction unit 6 corrects the positional deviation between or among the initial sound pressure distributions output from the first distribution obtaining unit 5, each of which is obtained for the corresponding pulsed light. More specifically, the positional deviation correction unit 6 carries out the correction of the positional deviation in such a manner that the distribution state of the photoacoustic wave generation source matches between or among the plurality of pieces of initial sound pressure distribution information. What is expressed by the positional deviation herein is occurrence of a deviation or a deformation between or among the plurality of obtained pieces of initial sound pressure distribution information, in particular, characteristic portions in images indicating the initial sound pressure distributions, due to the movement or the like of the measured object 2, as described above.

The positional deviation correction unit obtains the plurality of initial sound pressure distribution images (the plurality of pieces of initial sound pressure distribution information) obtained each time pulsed light is chronologically emitted. The positional deviation correction unit 6 carries out the correction of the positional deviation by setting, as a reference, the initial sound pressure distribution image with respect to the pulsed light with which the object 2 is irradiated first, and carrying out deformation registration on the initial sound pressure distribution image(s) obtained from pulsed light other than the one emitted first, based on the set reference. In tine present example, the image set as the reference has been described as the initial sound pressure distribution image with respect to the pulsed light with which the object 2 is irradiated first, but may be the initial sound pressure distribution image with respect to any pulsed light. The correction of the positional deviation is not limited to the method that corrects the positional deviation so as to align any one of the two initial sound pressure distribution images to the other as described above, and may be carried out by correcting both the pieces of distribution information (the distribution images) according to each other.

The method for the registration (positional deviation correction) can be embodied by employing a known method. In the present example, the positional deviation correction unit 6 prepares a reference image and a deformation image, and obtains deformation information between the reference image and the deformation image. This information means a relationship between a certain coordinate value in the reference image and a coordinate corresponding thereto in the deformation image. In other words, this information can be expressed as a function f that obtains, based on a certain coordinate value Xfix in the reference image, a coordinate value Xmov corresponding thereto in the deformation image as indicated by an expression 2.

Xmov=f(Xfix) Expression 2

This processing calculates a continuous deformation based on a plurality of corresponding points discretely existing in the space. This processing is performed with use of a known interpolation method. Examples thereof include a method using a radial basis function (RBF), and a method using a B-spline, which is called the free-form deformation (FFD) method. Further, the positional deviation correction unit 6 may carry out the deformation registration in a stepwise manner, such as using the FFD after a deviation has been roughly corrected using an affine transformation. The positional deviation correction unit 6 deforms the deformation image with use of the obtained function, and evaluates validity of the deformation with use of an evaluation function. Generally, an index indicating a degree of image match between the reference image and the deformation image, such as a normalized cross-correlation, is used therefor. Further, a deformation field of the target image can be obtained from the function f obtained in this manner. The deformation field here means a field of the deformation from the deformation image to the reference image. In the present example, the initial sound pressure distribution is directly used for the correction of the positional deviation, but may be subjected to preliminary processing, such as a removal of an unnecessary portion and a conversion into a logarithmic image of the initial sound pressure distribution. In the case where, for example, the logarithmic image is generated, the initial sound pressure distribution with its positional deviation corrected can be obtained applying the deformation field generated at the time of the correction of the positional deviation to the original initial sound pressure distribution. Then, the positional deviation correction unit 6 corrects the light quantity distribution information with use of the deformation field generated at the time of the correction of the positional deviation.

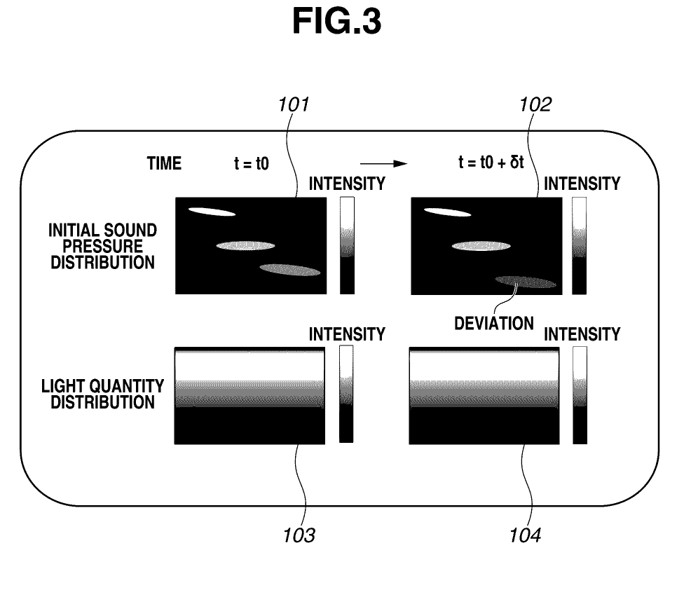

FIG. 3 is a schematic diagram illustrating the initial sound pressure distribution image and the light quantity distribution information about a corresponding pulsed light emitted to the object 2. FIG. 3 illustrates an initial sound pressure distribution image 101 at time t=t0, an initial sound pressure distribution image 102 at time t=t0+Δt, a light quantity distribution 103 at time t=t0, and a light quantity distribution 104 at time t=t0+Δt. Assume that the light quantity distribution is the same between both the times, and only a position of a circular absorbent located at a lowermost position in the initial sound pressure distribution image is different between them due to a deformation of the object 2. This absorbent itself is the same but is irradiated with different light quantities, and thus generates initial sound pressures at different intensifies. Accordingly, the intensity of the initial sound pressure is different at the absorbent.

If the positional deviation of this absorbent at time t=t0+Δt is corrected by the correction of the positional deviation based on the initial sound pressure distribution image 101 at time t=t0 as the reference,

this correction allows the positions of the absorbent in the initial sound pressure distributions (images) 101 and 102 at times t=t0 and t=t0+Δt, respectively, to match each other. However, if the calculation (the calculation to obtain the absorption coefficient) is made on the initial sound pressure distribution 102 at t=t0+Δt with its positional deviation corrected with use of the light quantity distribution 104 illustrated in the drawing (the same light quantity distribution between both the times) without any correction carried out thereon, a correct distribution cannot be obtained as the absorption coefficient distribution at time t=t0+Δt as described above. This is because the calculation is made on the initial sound pressure distribution information 102 with use of the uncorrected light quantity 104 despite the fact that the correction of the positional deviation is carried out on the initial sound pressure distribution information (image) 102. Accordingly, the light quantity distribution 104 to be used to make the calculation on the initial sound pressure distribution 102 at time t=t0+Δt should be corrected (deformed) according to the correction (the deformation field) obtained at the time of the correction of the positional deviation on the initial sound pressure distribution information 102.

To that end, the positional deviation correction unit 6 corrects the light quantity distribution information in correspondence with the correction (information indicating the deformation field) carried out on the initial sound pressure distribution information (image), thereby calculating the corrected light quantity distribution information. In the present exemplary embodiment, the positional deviation correction unit 6 corrects the light quantity distribution information in such a manner that the relationship between the distribution state of the photoacoustic wave generation source that is obtained by the first distribution obtaining unit 5 and the distribution state of the light quantity that is obtained by the light quantity distribution obtaining unit 4, and the relationship between the corrected distribution state of the photoacoustic wave generation source and the corrected distribution state of the light quantity match each other.

Then, the second distribution obtaining unit 7 obtains the absorption coefficient distribution by making the calculation on the initial sound pressure distribution information with use of the light quantity distribution information based on the corrected initial sound pressure distribution information and the corrected light quantity distribution information obtained in this manner, using the expression 1 (the Gruneisen constant in the expression 1 can be regarded constant).

Then, the obtained absorption coefficient distribution information is output to the display control unit 9.

In the present exemplary embodiment, the photoacoustic apparatus makes the calculation for obtaining the absorption coefficient distribution based on the corrected light quantity distribution when calculating the absorption coefficient distribution information in the object 2 from the chronologically obtained initial sound pressure distribution information, as described above. By using this method, the photoacoustic apparatus makes the calculation even on the initial sound pressure distribution information where the positional deviation occurs with use of the correct light quantity distribution information, and thus can obtain the accurate absorption coefficient distribution information (the characteristic information).

The display control unit 9 generates the image data to be displayed on the display unit 8, based on the initial sound pressure distribution information generated by the first distribution obtaining unit 5, and the distribution data indicating the distribution of the characteristic such as the absorption coefficient, which is generated by the positional deviation correction unit 6 and the second distribution obtaining unit 1. More specifically, the display control unit performs image processing such as a luminance conversion, a distortion correction, and logarithmic compression processing based on the distribution data. Further, the display control unit performs display control, such as presenting the display various kinds of display items arranged side by side together with the distribution data.

### (Flow of Processing Performed by Signal Processing Unit 40)

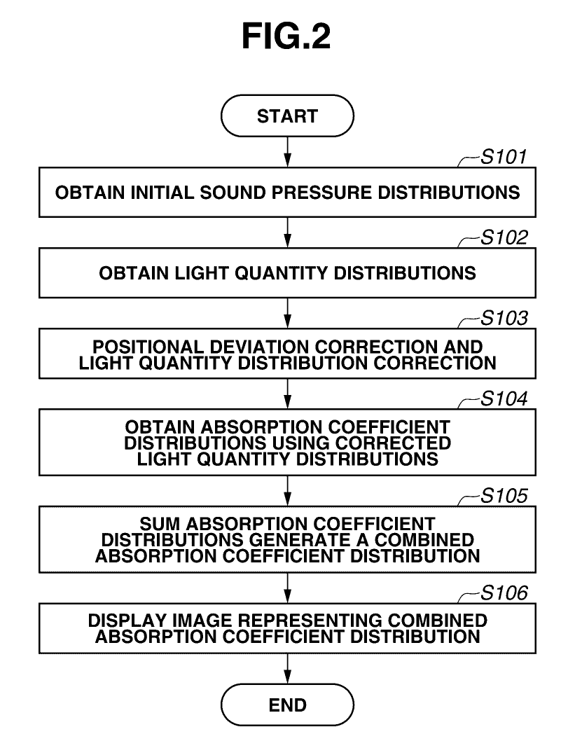

Next, a flow of processing performed by the signal processing unit 40 will be described. FIG. 2 is a flowchart illustrating the flow of the processing performed by the signal processing unit 40 according to one or more aspects of the present disclosure. The flow illustrated in FIG. 2 is started from a state in which the reception signal is sequentially input from the probe 30 to the signal collection unit 10 in the signal processing unit 40 for each of the pulses of the light with which the object 2 is irradiated, and is subjected to the processing such as the AD conversion and the amplification performed by the signal collection unit 10.

In step S101, the first distribution obtaining unit 5 obtains (calculates) the distribution information indicating the distribution state of the photoacoustic wave generation source in the object 2 with use of the electric signal obtained by receiving the photoacoustic wave generated from the object 2 irradiated with light having a wavelength λ₁. The object 2 is irradiated a plurality of times with the light having the wavelength λ₁ at different timings, and the first distribution obtaining unit 5 calculates the distribution information indicating the distribution state of the photoacoustic wave generation source for the corresponding pulsed light.

In step S102, the light quantity distribution obtaining unit 4 obtains the light quantity distribution information, indicating the distribution state of the light quantity in the object 2, obtained each time the object 2 is irradiated with the pulsed light of the wavelength λ₁. It is desirable that a region from which the light quantity distribution information is obtained matches a region from which the initial sound pressure distribution information is obtained, but the light quantity distribution information may be obtained from the entire region of the object 2. In this case, the light quantity distribution obtaining unit 4 generates the light quantity distribution corresponding to the initial sound pressure distribution cutting out the corresponding region.

In step S103, the positional deviation correction unit 6 corrects the obtained distribution information about the photoacoustic wave generation source, and also corrects the obtained light quantity distribution information in correspondence with this correction. More specifically, first, the positional deviation correction unit 6 carries out the correction of the positional deviation between or among the pieces of initial sound pressure distribution information obtained by the first distribution obtaining unit 5 each time the object 2 is irradiated with the pulsed light. In the correction of the positional deviation, the positional deviation correction unit 6 corrects the positional deviation by setting one piece of initial sound pressure distribution information between or among the plurality of pieces of initial sound pressure distribution information as the reference, and carrying out the deformation registration on the other piece(s) of initial sound pressure distribution information so as to register them to the initial sound pressure distribution information set as the reference. The positional deviation correction unit 6 uses the normalized cross-correlation as the evaluation value for the correction of the positional deviation, and deforms the image (s) with use of the spline. Then, the positional deviation correction unit 6 further carries out the correction of the positional deviation on the light quantity distribution information in correspondence with the correction used in the deformation registration on the initial sound pressure distribution information.

In step S104, the second distribution obtaining unit 7 makes the calculation on the initial sound pressure distribution information about the corresponding pulsed light with use of the light quantity distribution, thereby generating the absorption coefficient distribution. The light quantity distribution information used at this time is the light quantity distribution information corrected in correspondence with ne correction carried out on the initial sound pressure distribution information subjected to the correction of the positional deviation, as described above. In other words, the light quantity distribution information used to calculate the absorption coefficient distribution information is the light quantity distribution information deformed with use of the deformation field obtained at the time of the correction of the positional deviation based on the initial sound pressure distribution information se a as the reference. The initial sound pressure distribution information set as the reference is not deformed at the time of the correction of the positional deviation. Accordingly, the light quantity distribution information used to obtain the absorption coefficient distribution information with respect to the initial sound pressure distribution information set as the reference is the original light quantity distribution information obtained at the time of the irradiation without the correction carried out thereon.

In step S105, further, the second distribution obtaining unit 7 combines (integrates) the plurality of pieces of absorption coefficient distribution information with respect to the corresponding pulsed light obtained in step S104, thereby obtaining combined absorption coefficient distribution information.

In step S106, the display control unit 9 displays image data indicating the calculated combined (integrated) absorption coefficient distribution on the display unit 8.

In this manner, the present exemplary embodiment can improve the accuracy of the calculation of the absorption coefficient distribution to generate, even with respect to the initial sound pressure distribution information an the state subjected to the correction of the Positional deviation.

Further, in the above-described example, the second distribution obtaining unit 7 obtains the absorption coefficient distribution information as the characteristic distribution based on the optical absorption, but the present exemplary embodiment is not limited thereto and may obtain the “oxygen saturation distribution” or the like.

Further, in the above-described example, the target region has been described as the blood portion in the blood vessel by way of example, but the present exemplary embodiment is not limited thereto. The target region may be a blood vessel wall, a lymph duct, a muscle tissue, a mammary gland tissue, a fat tissue, and an aggregation of externally injected substances such as a molecular target drug as a radiopaque dye.

Next, a specific example of a configuration of each of the component units according to the present exemplary embodiment will be described.

### (Light Irradiation Unit 1)

Desirably, the light irradiation unit 1 is a pulsed light irradiation unit capable of generating pulsed light in the order of nanoseconds to microseconds. A pulse width of approximately 1 nanosecond or longer and 100 nanoseconds or shorter is used as a specific pulse width. Further, a wavelength in a range of 400 nm or longer and 1600 nm or shorter is used as the wavelength. Especially, in a case where the photoacoustic apparatus is expected to image a deep portion of a biological body, the light irradiation unit 1 uses light in a wavelength band called a “biological window” (a wavelength band less absorbable by a background tissue of the biological body). More specifically, a wavelength band of 700 nm or longer and 1100 nm or shorter is desirable in this case. On the other hand, in a case where the photoacoustic apparatus is expected to image a blood vessel near the surface of the biological body at a high resolution, it is desirable to use a visible light. However, a terahertz wave region, a microwave region, and a radio wave region can also be used.

Specifically, a laser is desirable as the light irradiation unit 1, and various types of lasers, such as a solid-state laser, a gas laser, a dye laser, and a semiconductor laser, can be used as the laser. Particularly, a pulsed laser such as a neodymium-doped yttrium aluminum garnet (Nd:YAG) laser and an alexandrite laser is desirable. Further, a titanium sapphire (Ti:sa) laser using Nd:YAG laser light as excitation light, and an optical parametric oscillator (OPO) laser may also be used as the light irradiation unit 1. Further, a light-emitting diode and the like can also be used instead of the laser.

Desirably, the pulsed light output from the light irradiation unit 1 is guided to the object 2 by a member (an optical member) that propagates light, such as an optical fiber, a lens, a mirror, and/or a diffusing plate. Further, a spot shape and a light density of the pulsed light can also be changed with use of these optical members when the pulsed light is guided.

### (Probe 30)

The probe 30 includes one or more conversion element(s) 3. Any conversion element may be used as the conversion element 3 as long as the conversion element is an element capable of receiving the photoacoustic wave and converting it into the electric signal, such as a piezoelectric element using the piezoelectric phenomenon such as lead zirconate titanate (PZT), a conversion element using optical resonance, and as electrostatic capacitance type conversion element such as a capacitive micromachined ultrasonic transducer (CMUT). In the case where the probe 30 includes the plurality of conversion elements 3, it is desirable that the plurality of conversion elements 3 is disposed so as to be arrayed on a flat surface or a curved surface, such as an array called a 1-dimensional (D) array, a 1.5-D array, a 1.75-D array, or a 2-D array.

Further, the probe 30 may be configured to be mechanically moveable relative to the object 2, or may be a hand-held type probe where a user holds and moves the probe 30. In the case where the photoacoustic apparatus is the photoacoustic microscope, it is desirable that the probe 30 is configured as the focusing probe and is mechanically moveable over the surface of the object 2. Further, it is desirable that the irradiation position of the irradiation light and the probe 30 are moveable in synchronization with each other. Further, the probe 30 may be equipped therein with an amplifier that amplifies the analog signal output from the conversion element 3.

### (Display Unit 8)

A display, such as a liquid crystal display (LCD), a cathode ray tube (CRT), and an organic electroluminescence (EL) display, can be used as the display unit 8. The object information obtaining apparatus according to the present exemplary embodiment may be configured in such a manner that the display unit 8 is unequipped therein, but is prepared separately and connected to the object information obtaining apparatus.

### (Signal Processing Unit 40)

A circuit generally called a data acquisition system (DAS) can be used for the signal collection unit 10. More specifically, the signal collection unit 10 includes an amplifier that amplifies the reception signal, an AD converter that digitizes the analog reception signal, a first-in-first-out (FIFO) that stores the reception signal, a memory such as a random access memory (RAM), and the like.

A processor, such as a central processing unit (CPU), a microprocessor unit (MPU), and a graphics processing unit (CPU), can be used as the first distribution obtaining unit 5, the positional deviation correction unit 6, and the second distribution obtaining unit 7. Further, an arithmetic circuit, such as a field-programmable gate array (FPGA) chip, may also be used as them. The first distribution obtaining unit 5, the positional deviation correction unit 6, and the second distribution obtaining unit 7 may be constructed with use of not only a single processor or arithmetic circuit but also a plurality of processors or arithmetic circuits.

Further, the first distribution obtaining unit 5, the positional deviation correction unit 6, and the second distribution obtaining unit 7 may include memory that stores the reception signal output from the signal collection unit 10. Typically, the memory includes a storage medium, such as a read only memory (ROM), a RAM, and a hard disk. The memory may include not only a single storage medium but also a plurality of storage media. Typically, the memory includes a storage medium/storage media such as one or more ROM(s), RAM(s), and hard disk(s).

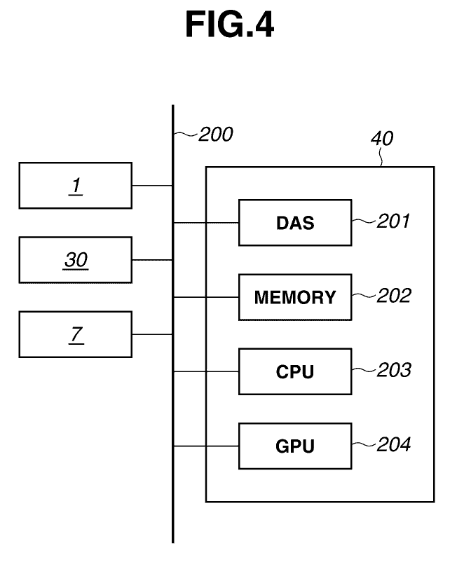

FIG. 4 is a schematic diagram illustrating a relationship between one specific example of the signal processing unit 40 and external devices in the example illustrated in FIG. 4, the signal processing unit 40 includes a DAS 201, a memory 202, a CPU 203, and a GPU 204.

The DAS 201 is in charge of one of functions of the signal collection unit 10 in the present exemplary embodiment. The digital signal transferred from the DAS 201 is stored in the memory 202.

The CPU 203 is in charge of a part of functions of the first distribution obtaining unit 5, the positional deviation correction unit 6, the display control unit 9, and the second distribution obtaining unit 7 in the present exemplary embodiment. More specifically, the CPU 203 controls each of the functional blocks via a system bus 200. Further, the CPU 203 can perform signal processing such as integration processing and correction processing on the digital signal stored in the memory 202. Further, the CPU 203 writes the digital signal after the signal processing into the memory 202 again, which is provided to be used for the GPU 204 to generate the distribution data.

The GPU 204 is in charge of a part of the functions of the first distribution obtaining unit 5, the positional deviation correction unit 6, the display control unit 9, and the second distribution obtaining unit 7 in the present exemplary embodiment. More specifically, the GPU 204 generates the characteristic distribution data with use of the digital signal subjected to the signal processing performed by the CPU 203 and written into the memory 202. Further, the GPU 204 can generate the image data by applying various kinds of image processing, such as the luminance conversion, the distortion correction, and segmentation of a target region, to the generated distribution data. Similar processing can also be realized by the CPU 203.

FIGS. 5A, 5B, 5C, 5D, 5E, and 5F illustrate the result of the validation using the present method, and can confirm advantageous effects of the present exemplary embodiment as described above.

Now, the present exemplary embodiment will be described referring to the specific exemplary experiment. In the present example, the phantom imitating a breast was used as the object 2. The object 2 was irradiated with the light via a holding member, made from polymethylpentene, which held the object 2, and the probe 30 received the photoacoustic wave via the holding member. The probe 30 was the 2-D probe including the plurality of conversion elements 3 responsive to a frequency band of 1 MHz+40%.

First, the object 2 was irradiated with pulsed light having a wavelength of 797 nm from light irradiation unit 1, and the probe 30 received the photoacoustic wave. The signal processing unit 40 configured as illustrated in FIG. 4 reconstructed the image with use of the universal back-projection based on the reception signal obtained from the reception. Further, in the present example, the photoacoustic apparatus irradiated a same position without performing scanning with the pulsed light. This irradiation allowed the photoacoustic apparatus to obtain each three-dimensional initial sound pressure distribution for the corresponding pulsed light at the same position. The obtained initial sound pressure distribution was reconstructed only at the region corresponding to the irradiation light distribution of the pulsed light, which was 160 voxels in depth, 160 voxels in width, and 200 voxels in height in the present example.

Next, the photoacoustic apparatus converted the photoacoustic signal, obtained each time the object 2 is irradiated with the pulsed light, into the electric signal, and carried out the correction of the positional deviation between the individual pieces of initial sound pressure distribution information, each of which was obtained from this electric signal. In the case where the object 2 is the biological body, the spatial position of the absorbent (the blood in the present example) may change over time due to the breathing or the movement of the body. The initial sound pressure distribution information obtained each time the pulsed light is emitted indicates the initial sound pressure distribution around a time when the object 2 is irradiated with the pulsed light. Accordingly, if each position of the absorbent changes over time, calculating an arithmetic mean or the like of the pieces of initial sound pressure distribution information obtained each time the pulsed light is emitted without any correction carried out thereon results in an output of the integrated initial sound pressure distribution calculated with low accuracy.

The initial sound pressure distribution information based on the pulsed light with which the object 2 had been irradiated first is specified as the reference information (the reference image in the present example) between the pieces of initial sound pressure distribution information obtained each time the pulsed light is emitted. The photoacoustic apparatus deformed and registered the initial sound pressure distribution based on pulsed light other than the pulsed light first emitted according to this reference image, thereby correcting the positional deviation between both the pieces of initial sound pressure distribution information (the initial sound pressure distribution images). In the correction of the positional deviation, the photoacoustic apparatus deformed and registered the initial sound pressure distribution information by carrying out the FFD with use of the normalized cross-correlation. In the present example, since the object 2 was the phantom imitating the breast, a pressure was applied to the phantom from one side to deform it, thereby causing the positional deviation, after the phantom was irradiated with the pulsed light for the first time and before the phantom was irradiated with the next pulsed light.

Then, the photoacoustic apparatus calculated the pieces of absorption coefficient distribution information by making the calculation on the pieces of initial sound pressure distribution information, obtained each time the pulsed light is emitted, that had been registered to each other or one another while using the pieces of light quantity distribution information and also using the Gruneisen constant in the present example. As the light quantity distribution information, the photoacoustic apparatus corrected the light quantity distribution information to be used in the calculation made on the initial sound pressure distribution information subjected to the correction of the positional deviation, according to the correction of the positional deviation that had been carried out on the initial sound pressure distribution information. This correction allowed the photoacoustic apparatus to accurately obtain the absorption coefficient distribution information obtained each time the pulsed light is emitted.

Next, the photoacoustic apparatus integrated (combined) the absorption coefficient distributions obtained each time the pulsed light is emitted, thereby calculating the combined absorption coefficient distribution. More specifically, the photoacoustic apparatus performed the processing for calculating the arithmetic mean of the individual pieces of absorption coefficient distribution information, thereby obtaining the combined absorption coefficient distribution information.

In the present example, the photoacoustic apparatus has been described as obtaining the combined absorption coefficient distribution information, but may generate only the absorption coefficient distribution information obtained each time the pulsed light is emitted before the combination. With this configuration, the photoacoustic apparatus in the present example makes the calculation with use of the light quantity distribution information corresponding to the positional deviation even when the positional deviation occurs between or among the pieces of initial sound pressure distribution information obtained each time the pulsed light is emitted, and therefore can calculate the accurate absorption coefficient distribution information.

Next, a second exemplary embodiment will be described. The object information obtaining apparatus (e.g., photoacoustic apparatus) according to the present exemplary embodiment employs a substantially similar apparatus configuration to the object information obtaining apparatus according to the first exemplary embodiment, and thus a detailed description of each component will be omitted below. However, processing performed by the signal processing unit 40 has some difference from the first exemplary embodiment, and thus, the present exemplary embodiment will be described below only focusing on the difference from the first exemplary embodiment.

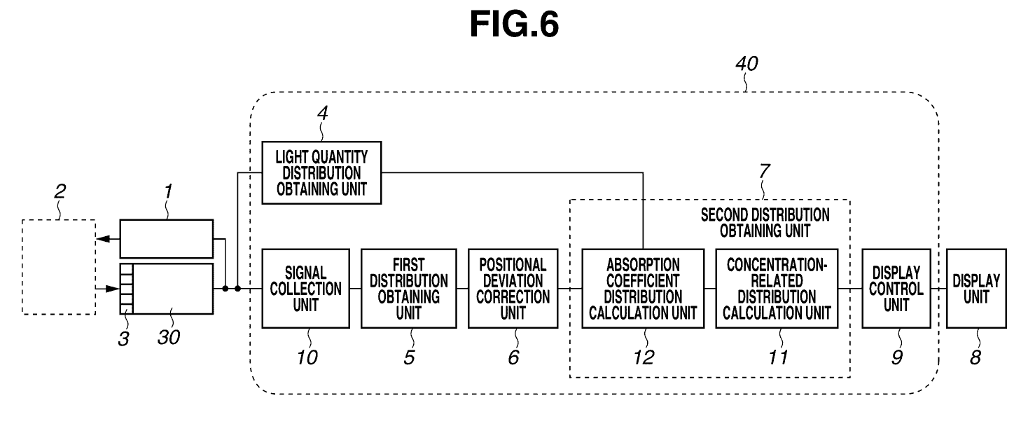

FIG. 6 is a schematic diagram illustrating a configuration of the photoacoustic apparatus according to one or more aspects of the present disclosure.

The photoacoustic apparatus according to the present exemplary embodiment obtains the absorption coefficient distribution information with use of the initial sound pressure distribution information subjected to the correction of the positional deviation and the light quantity distribution information also subjected to the correction of the positional deviation in a similar manner, in a case where the positional deviation occurs due to the movement of the object body between or among the pieces of initial sound pressure distribution information generated from the pulsed of the mutually different wavelengths between measurements on a same position. Then, the photoacoustic apparatus according to the present exemplary embodiment obtains the oxygen saturation distribution calculated with improved accuracy from the generated absorption coefficient distributions calculated improved accuracy. Thus, the second distribution obtaining unit includes a concentration-related distribution calculation unit 11.

### (Concentration-Related Distribution Calculation Unit 11)

The concentration-related distribution calculation unit 11 calculates the concentration-related distribution from the pieces of distribution information derived from the plurality of wavelengths that are generated by an absorption coefficient distribution calculation unit 12 in the following description, a description is given of an example in which the oxygen saturation distribution is obtained as the concentration-related distribution.

Assuming that optical absorption other than absorption by hemoglobin is negligibly small at the wavelength λ₁ and a wavelength λ₂, absorption coefficients at the wavelength λ₁ and the wavelength λ₂ are expressed as indicated by expressions 3 and 4, respectively, with use of a molar absorption coefficient of oxyhemoglobin and a molar absorption coefficient of deoxyhemoglobin.

μₐ (λ₁)=εₒₓ (λ₁)Cₒₓ+ε_{de} (λ₁)C_{de} Expression 3

μₐ (λ₂)=εₒₓ (λ₂)Cₒₓ+ε_{de} (λ₂)C_{de} Expression 4

In these expressions 3 and 4, μₐ(λ₁) represents the absorption coefficient regarding the light having the wavelength λ₁ at the position (i, j, k), and μₐ (λ₂) represents the absorption coefficient regarding the light having the wavelength λ₂ at the position (i, j, k), which can be expressed in units of [mm⁻¹]. Cₒₓ represents an amount of oxyhemoglobin [mol], and C_{de} represents an amount of deoxy hemoglobin [mol]. Assume that both of them represent a value at the position (i, j, k).

Further, εₒₓ (λ₁) and ε_{de} (λ₁) represent the molar absorption coefficients [mm⁻¹mol⁻¹] of the oxyhemoglobin and the deoxyhemoglobin at the wavelength λ₁, respectively. Further, εₒₓ (λ₂) and ε_{de} (λ₂) represent the molar absorption coefficients [mm⁻¹mol⁻] of the oxyhemoglobin and the deoxvhemoglobin at the wavelength λ₂, respectively. The molar absorption coefficients εₒₓ (λ₁), ε_{de} (λ₁), εₒₓ (λ₂), and ε_{de} (λ₂) can be obtained in advance by measuring them or referring to literature data.

Accordingly, Cₒₓ and C_{de} are each obtained by solving the expressions 3 and 4 as simultaneous equations with use of the molar absorption coefficients, μₐ (λ₁), and μₐ (λ₂). In a case where the photoacoustic apparatus uses a large number of wavelengths, the case can be handled by employing the least square method. Further, oxygen saturation SO₂ is defined to be a ratio of the amount of oxyhemoglobin in the total amount of hemoglobin, as indicated by an expression 5. Thus, the oxygen saturation SO₂ can be expressed by an expression 6 based on the expressions 3, 4, and 5.

Accordingly, the concentration-related distribution calculation unit 11 can obtain the oxygen saturation SO₂ at the position (i, j, k) based on the molar absorption coefficients, μₐ (λ₁) and μₐ (λ₂) with use of the expression 6.

$$\begin{matrix} [ Mathematical Expression 5 ] & \\ {SO}_{2} = \frac{{C}_{ox}}{{C}_{ox} + {C}_{de}} & Expression 5 \\ [ Mathematical Expression 6 ] & \\ {SO}_{2} = \frac{\frac{{μ}_{a} ( {λ}_{2} )}{{μ}_{a} ( {λ}_{1} )} {ɛ}_{de} ( {λ}_{1} ) - {ɛ}_{de} ( {λ}_{2} )}{( {ɛ}_{ox} ( {λ}_{2} ) - {ɛ}_{de} ( {λ}_{2} ) ) - \frac{{μ}_{a} ( {λ}_{2} )}{{μ}_{a} ( {λ}_{1} )} ( {ɛ}_{ox} ( {λ}_{1} ) - {ɛ}_{de} ( {λ}_{1} ) )} & Expression 6\end{matrix}$$

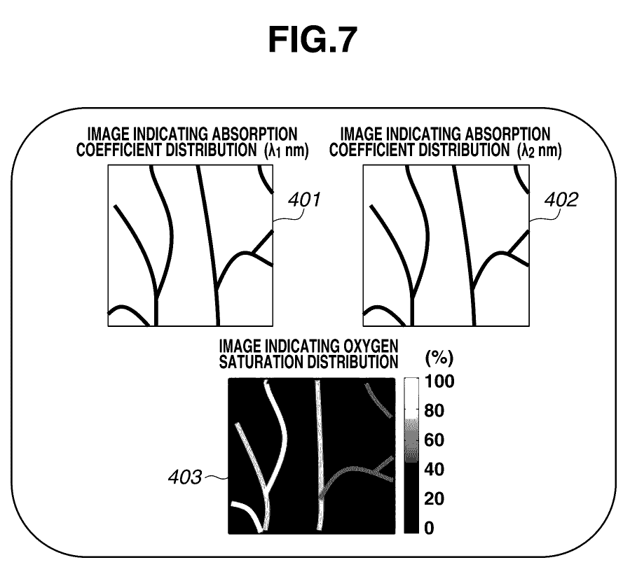

The concentration-related distribution calculation unit 11 can obtain the oxygen saturation at each position by performing this processing with respect to each position, so that the oxygen saturation distribution can be obtained. FIG. 7 illustrates one example of a display screen in a case where the oxygen saturation distribution is obtained from the absorption coefficient distribution at the wavelength λ₁ and the absorption coefficient distribution at the wavelength λ₂. This display screen displays respective images of an absorption coefficient distribution 401 at the wavelength λ₁, an absorption coefficient distribution 402 at the wavelength and an oxygen saturation distribution 40. The oxygen saturation distribution may be three-dimensional distribution data (data formed from a collection of voxels) corresponding to a certain region in the object 2, or may be two-dimensional distribution data (data formed from a collection of pixels) corresponding to one cross section thereof. In FIG. 7, the oxygen saturation value at a region where no absorbent exists is indicated as 0% for the sake of convenience. Further, the oxygen saturation distribution is obtained in the form of the ratio of the absorption coefficient distributions, and can be appropriately obtained as long as the absorption coefficient distributions at the plurality of wavelengths are relatively correct. Accordingly, the absorption coefficient distributions do not necessarily have to be obtained correctly as absolute values.

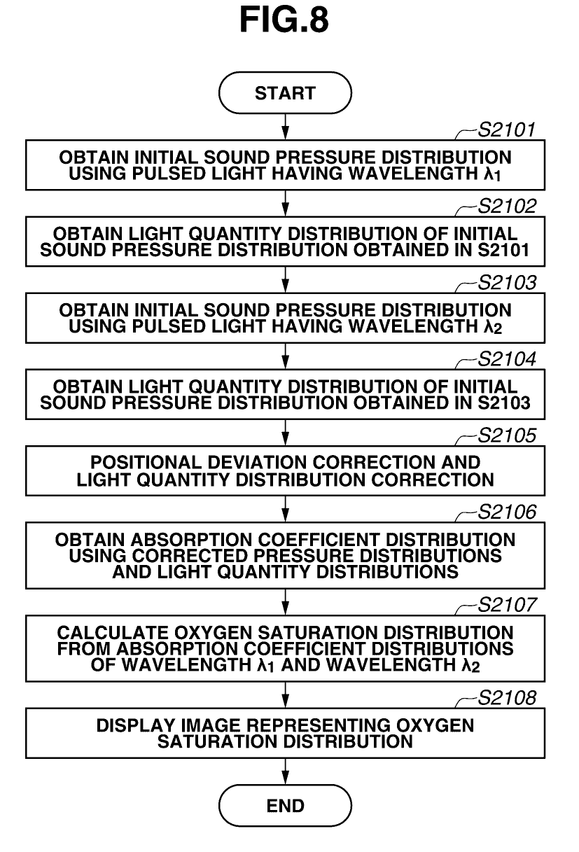

FIG. 8 is a flowchart illustrating a flow of the processing performed by the signal processing unit 40 according to one or more aspects of the present disclosure. The flow illustrated in FIG. 8 is started from the state in which the reception signal is sequentially input from the probe 30 to the signal collection unit 10 in the signal processing unit 40 for each of the pulses of the light with which the object 2 is irradiated, and is subjected to the processing such as the AD conversion and the amplification performed by the signal collection unit 10.

In step S2101, the first distribution obtaining unit 5 calculates the initial sound pressure distribution information with respect to the pulsed light having the wavelength λ₁ with use of the reception signal derived from the pulsed light having the wavelength λ₁. In other words, the first distribution obtaining unit obtains first distribution information indicating the distribution of the photoacoustic wave generation source in the object 2, which is obtained with use of light having a first wavelength.

In step S2102, the light quantity distribution obtaining unit 4 obtains the light quantity distribution information in the object 2 derived from the pulsed light having the wavelength λ₁ with which the object 2 has been irradiated. In other words, the light quantity distribution obtaining unit 4 obtains the light quantity distribution information indicating the distribution state of the light quantity in the object 2.

In step S2103, the first distribution obtaining unit 5 calculates the initial sound pressure distribution information with respect to the pulsed light having the wavelength λ₂ with use of the reception signal derived from the pulsed light having the wavelength λ₂, which is a different wavelength from the wavelength λ₁, as in step S2101. In other words, with step S2013, the first distribution obtaining unit 5 obtains second distribution information indicating the distribution of the photoacoustic wave generation source in the object 2, which is obtained with use of light having a second wavelength different from the first wavelength.

In step S2104, the light quantity distribution obtaining unit 4 obtains the light quantity distribution information in the object 2 derived from the pulsed light having the wavelength λ₂ with which the object 2 has been irradiated, as in step S2102.

In step S2105, the positional deviation correction unit 6 corrects the obtained distribution information about the photoacoustic wave generation source, and also corrects the obtained light quantity distribution information in correspondence with the correction. More specifically, first, the positional deviation correction unit 6 carries out the correction of the positional deviation between the initial sound pressure distribution information with respect to the pulsed light having the wavelength λ₁ that has been obtained in step S2101, and the initial sound pressure distribution information with respect to the pulsed light having the wavelength λ₂ that has been obtained in step S2103. In the correction of the positional deviation, the positional deviation correction unit 6 corrects the positional deviation by setting the initial sound pressure distribution information with respect to the pulsed light having the wavelength λ₁ as the reference, and carrying out he deformation registration on the initial sound pressure distribution information with respect to the pulsed light having the wavelength λ₂ based on the set reference. In other words, this step means that the positional deviation correction unit 6 performs the processing for correcting the second distribution information in such a manner that a position of a specific photoacoustic wave generation source indicated in the second distribution information (S2103) approaches the position of the specific photoacoustic wave generation source indicated in the first distribution information (S2101). The positional deviation correction unit 6 uses the normalized cross-correlation as the evaluation value for evaluating the correction of the positional deviation, and employs the FFD. In the present example, the positional deviation correction unit 6 carries out the correction of the positional deviation between the two wavelengths, but may carry out the correction of the positional deviation not only between two wavelengths but also among a plurality of wavelengths. In this case, the positional deviation correction unit 6 sets the initial sound pressure distribution of any of the wavelengths as the reference, and carries out the deformation registration on the initial sound pressure distributions of the other wavelengths according to the reference. Then, the positional deviation correction unit 6 further carries out the correction of the positional deviation on the light quantity distribution information with use of the correction used in the deformation registration on the initial sound pressure distribution information. In other words, the positional deviation correction unit 6 corrects the light quantity distribution information according to the correction processing performed in such a manner that the position of the specific photoacoustic wave generation source indicated in the second distribution information approaches the position of the specific photoacoustic wave generation source indicated in the first distribution information. More specifically, in the present exemplary embodiment, the positional deviation correction unit 6 carries out the correction of the positional deviation on the initial sound pressure distribution information with respect to the pulsed light having the wavelength λ₂ as described above, and therefore carries out the correction of the positional deviation on the light quantity distribution information obtained in step S2104. In other words, the light quantity distribution information includes first light quantity distribution information corresponding to the light having the first wavelength and second light quantity distribution information corresponding to the light having the above-described second wavelength, but the light quantity distribution information to be corrected at this time is the second light quantity distribution. information, which is the information obtained in step S2104.

In step S2106, the second distribution obtaining unit 7 generates the absorption coefficient distribution. obtained by irradiating the object 2 with the pulsed light of mutually different wavelengths between measurements with use of the initial sound pressure distribution information and the light quantity distribution correction information about each wavelength that have been obtained from the execution of the steps as far as the above-described step, step S2105, and the Gruneisen constant. As described above, the initial sound pressure distribution information and the light quantity distribution information derived from the irradiation with the pulsed light having the wavelength λ₂ that have been obtained in step S2105 are the distribution information subjected to the correction of the positional deviation. In other words, the second distribution obtaining unit 7 obtains the information regarding the characteristics in the object 2 with the use of the second distribution information subjected to the correction processing (step S2103) and, the corrected light quantity distribution information (step S2104). As described above, the initial sound pressure distribution information with respect to the pulsed light having the wavelength λ₁ is set as the reference, so that the correction of the positional deviation is not carried out on the initial sound pressure distribution information with respect to the pulsed light having the wavelength λ₁ that has been obtained in step S2101, and the light quantity distribution information in the object 2 with respect to the pulsed light having the wavelength λ₁ that has been obtained in step S2102. In other words, this corresponds to obtaining first characteristic information in the object 2 with use of the first distribution information (S2101) and the light quantity distribution information (S2102), and obtaining second characteristic information regarding the characteristics in the object 2 with use of the second distribution information subjected to the correction processing (2105) and he corrected light quantity distribution information (S2105).

In step S2107, the second distribution obtaining unit 7 further calculates the oxygen saturation with use of the above-described expression 6 from the absorption coefficient distribution of each of the wavelengths that has been subjected to the correction of the positional deviation, which has been obtained in step S2016. In other words, the second distribution obtaining unit 7 obtains the information regarding the object 2 with use of the above-described first characteristic information and second characteristic information.

In step S2108, the display control unit 9 generates image data based on the data indicating the oxygen saturation distribution output from the concentration-related distribution calculation unit 11, and displays the generated image data on the display unit 8.

In this manner, the present exemplary embodiment allows the photoacoustic apparatus to, even in a case where the positional deviation occurs between or among the initial sound pressure distributions of the plurality of wavelengths, carry out the correction of the positional deviation between or among the wavelengths and obtain the further accurate oxygen saturation distribution from the light quantity distribution (s) corrected in consideration of the deviation.

In the above-described example, the concentration-related distribution calculation unit 11 included in the second distribution obtaining unit obtains the oxygen saturation distribution as the concentration-related distribution, but the present exemplary embodiment is not limited thereto. As described above, the concentration-related distribution may be any distribution as long as the distribution is a “distribution of a value related to a concentration of a substance (the concentration-related distribution)” obtainable with use of the “characteristic distribution based on the optical absorption” with respect to the plurality of wavelengths. More specifically, the concentration-related distribution may be distribution information such as a “weighed oxygen saturation value”, “total hemoglobin concentration”, an “oxyhemoglobin concentration”, a “deoxyhemoglobin concentration”, a “glucose concentration”, a “collagen concentration”, a “melanin concentration”, and a “volume fraction” of fat or water.

Further, in the above-described example, the second distribution obtaining unit 7 obtains the absorption coefficient distribution as the characteristic distribution based on the optical absorption, but the present exemplary embodiment is not limited thereto and may obtain the “sound pressure distribution (typically, the initial sound pressure distribution)”. For example, since μₐ can be expressed as P/(Γ·φ) from the expression 1, substituting P/(Γ·φ) for μₐ in the expression 5 allows the oxygen saturation to be directly calculated from the initial sound pressure. In other words, the distribution obtaining unit can directly obtain the oxygen saturation distribution from the data indicating the initial sound pressure distribution even without first obtaining the absorption coefficient distribution after obtaining the initial sound pressure distribution.

Next, a third exemplary embodiment will be described. The third exemplary embodiment is an exemplary embodiment that is a combination of the first exemplary embodiment and the second exemplary embodiment. Accordingly, the object information obtaining apparatus according to the present exemplary embodiment employs a similar apparatus configuration to the object information obtaining apparatuses according to the first and second exemplary embodiments, and thus a detailed description of each component will be omitted below. However, processing performed by the signal processing unit 40 has some difference from the first and second exemplary embodiments, so that the present exemplary embodiment will be described below only focusing on the difference.

The photoacoustic apparatus according to the present exemplary embodiment carries out the correction of the positional deviation according to the first exemplary embodiment, and obtains the pieces of absorption coefficient distribution information combined for the individual wavelengths, further corrects the positional deviation between or among the pieces of combined absorption coefficient distribution information of the individual wavelengths, and obtains the oxygen saturation distribution information with use of the pieces of combined absorption coefficient distribution information registered to each other or one another. With this configuration, the photoacoustic apparatus can obtain absorption coefficient distributions and the oxygen saturation distribution calculated with improved accuracy.

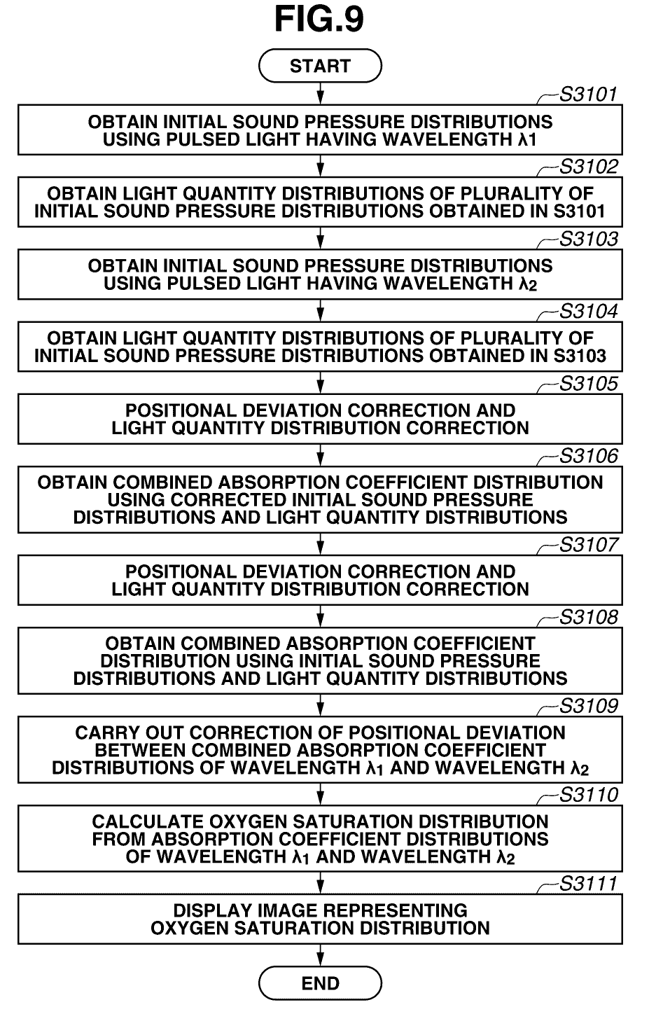

FIG. 9 is a flowchart illustrating a flow of the processing performed by the signal processing unit 40 according to one or more aspects of the present disclosure.

In step S3103, the first distribution obtaining unit 5 calculates the pieces of initial sound pressure distribution information with respect to the pulsed light of the wavelength λ₁ emitted a plurality of times with use of the reception signals derived from the pulsed light of the wavelength λ₁ emitted a plurality of times toward the object 2, as in step S101 in the first exemplary embodiment.

In step S3102, the light quantity distribution obtaining unit 4 obtains the light quantity distribution information in the object 2 derived from the pulsed light having the wavelength λ₁ with which the object 2 has been irradiated, as in step S102 in the first exemplary embodiment.

In step S3103, the first distribution obtaining unit 5 calculates the pieces of initial sound pressure distribution information with respect to the pulsed light of the wavelength λ₂ emitted a plurality of times with use of the reception signals derived from the pulsed light of the wavelength λ₂ emitted a plurality of times, which is the different wavelength from the wavelength λ₁, in step S101 in the first exemplary embodiment.

In step S3104, the light quantity distribution obtaining unit 4 obtains the light quantity distribution information in the object 2 derived. from the pulsed light having the wavelength λ₂ with which the object 2 has been irradiated, as in step S102 in the first exemplary embodiment.

In step S3105, the positional deviation correction unit carries out the correction of the positional deviation between or among the pieces of initial sound pressure distribution information with respect to the pulsed light of the wavelength λ₁ emitted a plurality of times, calculated in step S3101. Further, the positional deviation correction unit 6 also corrects the light quantity distribution information according to the content of this correction of the positional deviation.

In step S3106, the second distribution obtaining unit 7 generates the plurality of pieces of absorption coefficient distribution information with respect to pulsed light of the wavelength λ₁ emitted a plurality of times from a result of the correction of the positional deviation in step S3105 with use of the pieces of initial sound pressure distribution information and the pieces of light quantity distribution information with respect to the pulsed light of the wavelength λ₁ emitted a plurality of times that have been subjected to the correction of the positional deviation. Then, the second distribution obtaining unit 7 performs the processing for calculating the arithmetic mean of the plurality of pieces of absorption coefficient distribution information, thereby generating the combined absorption coefficient distribution information with respect to the wavelength λ₁, as in the first exemplary embodiment.

In step S3107, the positional deviation correction unit 6 carries the correction of the positional deviation between or among the pieces of initial sound pressure distribution information with respect to pulsed light of the wavelength λ₂ emitted a plurality of times, calculated in step S3103. Further, the positional deviation correction unit 6 also corrects the light quantity distribution information according to the correction of the positional deviation.

In step S3108, the second distribution obtaining unit 7 generates the plurality of pieces of absorption coefficient distribution information with respect to the pulsed light of the wavelength λ₂ emitted a plurality of times from a result of the correction of the positional deviation in step S3107 with use of the pieces of initial sound pressure distribution information and the pieces of light quantity distribution information with respect to the pulsed light of the wavelength λ₂ emitted a plurality of times that have been subjected to the correction of the positional deviation. Then, the second distribution obtaining unit 7 performs the processing for calculating the arithmetic mean of the plurality of pieces of absorption coefficient distribution information, thereby generating the combined absorption coefficient distribution information with respect to the wavelength λ₂, as in the first exemplary embodiment.

In step S3109, the positional deviation correction unit 6 further carries out the correction of the positional deviation between the combined absorption coefficient distributions of the wavelength λ₁ and the wavelength λ₂ that have been obtained in steps S3106 and S3108, respectively.

In step S3110, the second distribution obtaining unit 7 calculates the oxygen saturation distribution from the combined absorption coefficient distributions of the wavelength λ₁ and the wavelength λ₂. In other words, the second distribution obtaining unit 7 obtains the oxygen saturation distribution information, which is the concentration-related distribution information, with use of the plurality of pieces of combined absorption coefficient distribution information obtained by irradiating the object with light of mutually different wavelengths between measurements on a same position.

In step S3111, the display control unit generates the image data based on the data indicating the oxygen saturation distribution output from the concentration-related distribution calculation unit 11, and displays the generated image data on the display unit 8.

In the above-described manner, in the present exemplary embodiment, the photoacoustic apparatus generates absorption coefficient distribution information combined for one wavelength, and further carries out the correction of the positional deviation between or among the pieces of combined absorption coefficient distribution information of the plurality of wavelengths. At this time, even in a case where the positional deviation occurs between or among the pieces of initial sound pressure distribution information about the individual pluses with respect to each of the wavelengths, the photoacoustic apparatus performs the appropriate calculation processing based on the light quantity distribution information corrected in consideration of this positional deviation, which allows the photoacoustic apparatus to obtain the absorption coefficient distributions and oxygen saturation distribution calculated with improved accuracy. In the above-described example, the photoacoustic apparatus carries out the correction of the positional deviation between or among the pieces of initial sound pressure distribution information about the individual pulses having the same single wavelength, obtains the plurality of pieces of absorption coefficient distribution information with respect to the single wavelength with use of the pieces of light quantity distribution information subjected to the similar correction of the positional deviation, and generates the combined absorption coefficient distribution information by combining them. After that, the photoacoustic apparatus carries out the correction of the positional deviation between or among pieces of absorption coefficient distribution information of the plurality of wavelengths. However, the order in which the corrections of the positional deviation are carried out is not limited to this example. The corrections of the positional deviation may be carried out with use of the following method. After carrying out the correction of the positional deviation between or among the pieces of initial sound pressure distribution information about the plurality of pulses having the same single wavelength, the photoacoustic apparatus holds information indicating this positional deviation, and also carries out the correction of the positional deviation between or among the pieces of initial sound pressure distribution information about the plurality of pulses for another wavelength. Next, the photoacoustic apparatus further carries out the correction of the positional deviation between or among the pieces of initial sound pressure distribution information after the correction of the positional deviation for each of the wavelengths. After carrying out the corrections of the positional deviation in these two steps, the photoacoustic apparatus makes the calculation for obtaining the absorption coefficient distribution information with use of the light quantity distribution information subjected to the correction of the positional deviation to generate the absorption coefficient distribution integrated for each of the wavelengths, and calculates the oxygen saturation distribution. This method means that the photoacoustic apparatus carries out the correction of the positional deviation for each of the wavelengths and then further completes the correction of the positional deviation between or among the wavelengths as well, i.e., carries out all the corrections of the positional deviation regarding the initial sound pressure distribution information, and, after that, obtains the absorption coefficient distribution information with use of the light quantity distribution information subjected to the correction of the positional deviation and further obtains the oxygen saturation distribution information.

### Other Embodiments

Embodiment (s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment (s) and/or controlling the one or more circuits to perform the functions of one or more of the above-described embodiment(s). The computer may comprise one or more processors (e.g., central processing unit (CPU), micro processing unit (MPU)) and nay include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

The present disclosure can improve the accuracy of the calculation of the distribution information regarding the optical characteristic value in the object.

While the present disclosure has been described with reference to exemplary embodiments, the scope of the following claims are to be accorded the broadest interpretation so as to encompass all such modifications and equivalent structures and functions.

This application claims the benefit of Japanese Patent Application No. 2015-240582, filed December 9, 2015, which is hereby incorporated by reference herein in its entirety.

What is claimed is:

1. An object information obtaining apparatus comprising:
a light irradiation unit;
a conversion element configured to receive a photoacoustic wave generated in an object in response to irradiation with light from the light irradiation unit and convert the received photoacoustic wave into an electric signal;
a light quantity distribution obtaining unit configured to obtain light quantity distribution information representing a distribution state of a light quantity in the object with respect to the light with which the object is irradiated from the light irradiation unit;
first distribution obtaining unit configured to obtain distribution information representing a distribution state of a photoacoustic wave generation source in the object by using the electric signal;
a correction unit configured correct the distribution information obtained by the first distribution obtaining unit, and also correct the light quantity distribution information obtained by the light quantity distribution obtaining unit in correspondence with the correction; and
a second distribution obtaining unit configured to obtain characteristic distribution information in the object by using the corrected distribution information and the corrected light quantity distribution information.

2. The object information obtaining apparatus according to claim 1,
wherein the light irradiation unit irradiates the object a plurality of times with light at different timings,
wherein the first distribution obtaining unit obtains a plurality of pieces of distribution information, each of which is obtained for a corresponding irradiation with the light from the light irradiation unit, and
wherein the correction unit corrects at least one of the plurality of pieces of distribution information in such a manner that the distribution state of the photoacoustic wave generation source matches between two of the plurality of pieces of distribution information.

3. The object information obtaining apparatus according to claim 1,
wherein the light irradiation unit irradiates the object a plurality of times with light at different timings,
wherein the first distribution obtaining unit obtains a plurality of pieces of distribution information, each of which is obtained for a corresponding irradiation with the light from the light irradiation unit, and
wherein the correction unit corrects at least one of the plurality of pieces of distribution information in such a manner that the distribution state of the photoacoustic wave generation source matches between two of the plurality of pieces of distribution information.

4. The object information obtaining apparatus according to claim 1, wherein the correction unit corrects the light quantity distribution information in such a manner that a positional relationship between the distribution state of the photoacoustic wave generation source that is obtained by the first distribution obtaining unit and the distribution state of the light quantity that is obtained by the light quantity distribution obtaining unit, and a positional relationship between the corrected distribution state of the photoacoustic wave generation source and the corrected distribution state of the light quantity match each other.

5. The object information obtaining apparatus according to claim 1, wherein the characteristic distribution information is absorption coefficient distribution information in the object.

6. The object information obtaining apparatus according to claim 5, wherein the second distribution obtaining unit combines a plurality of pieces of absorption coefficient distribution information to obtain combined absorption coefficient distribution information.

7. The object information obtaining apparatus according to claim 6, wherein the second distribution obtaining unit obtains concentration distribution information by using plurality of pieces of combined absorption coefficient distribution information obtained by irradiating light of mutually different wavelengths between the plurality of pieces of combined absorption coefficient distribution information.

8. The object information obtaining apparatus according to claim 5, wherein the second distribution obtaining unit obtains concentration distribution information by using a plurality of pieces of absorption coefficient distribution information obtained by irradiating light of mutually different wavelengths between the plurality of pieces of absorption coefficient distribution information.

9. The object information obtaining apparatus according to claim 8, wherein the concentration distribution information is oxygen saturation distribution information.

10. An object information obtaining method comprising:
obtaining light quantity distribution information representing a distribution state of a light quantity in an object with respect to light with which the object is irradiated;
obtaining distribution information representing a distribution state of a photoacoustic wave generation source in the object by using an electric signal obtained by receiving a photoacoustic wave generated from the object irradiated with the light;
correcting the obtained distribution information, and also correcting the obtained light quantity distribution information in correspondence with the correction; and
obtaining characteristic distribution information in the object by using the corrected distribution information and the corrected light quantity distribution information.

11. An information obtaining method for obtaining information regarding a characteristic in an object by using a photoacoustic signal generated in response to irradiation of the object with light, the method comprising:
obtaining first distribution information representing a distribution of a photoacoustic wave generation source in the object that is obtained by using light having a first wavelength;
obtaining second distribution information representing the distribution of the photoacoustic wave generation source in the object that is obtained by using light having a second wavelength different from the first wavelength;
obtaining light quantity distribution information representing a distribution state of a light quantity in the object;
performing correction processing for correcting the second distribution information in such a manner that a position of a specific photoacoustic wave generation source in the second distribution information approaches a position of the specific photoacoustic wave generation source in the first distribution information;
correcting the light quantity distribution information in correspondence with the correction processing; and
obtaining the information regarding the characteristic in the object by using the second distribution information subjected to the correction processing and the corrected light quantity distribution information.

12. The information obtaining method according to claim 11, further comprising obtaining first characteristic information in the object by using the first distribution information and the light quantity distribution information, obtaining second characteristic information regarding the characteristic in the object by using the second distribution information subjected to the correction processing and the corrected light quantity distribution information, and further obtaining information regarding the object by using the first characteristic information and the second characteristic information.

13. The information obtaining method according claim 11, wherein the light quantity distribution information includes first light quantity distribution information corresponding to the light having the first wavelength and second light quantity distribution information corresponding to the light having the second wavelength, and the corrected light quantity distribution information is the corrected second light quantity distribution information.

14. An object information obtaining apparatus comprising:
a first distribution obtaining unit configured to obtain distribution information representing a distribution state of a photoacoustic wave generation source in an object by using an electric signal based on a photoacoustic wave generated in the object in response to irradiation with light;
a light quantity distribution obtaining unit configured to obtain light quantity distribution information representing a distribution state of a light quantity in the object with respect to the light with which the object is irradiated;
a correction unit configured to correct the distribution information obtained by the first distribution obtaining unit, and also correct the light quantity distribution information obtained by the light quantity distribution obtaining unit in correspondence with the correction; and
a second distribution obtaining unit configured to obtain characteristic distribution information in the object by using the corrected distribution information and the corrected light quantity distribution information.

15. The object information obtaining apparatus according to claim 14, wherein the correction unit corrects the light quantity distribution information in such a manner that a positional relationship between the distribution state of the photoacoustic wave generation source that is obtained by the first distribution obtaining unit and the distribution state of the light quantity that is obtained by the light quantity distribution obtaining unit, and a positional relationship between the corrected distribution state of the photoacoustic wave generation source and the corrected distribution state of the light quantity match each other.

16. The object information obtaining apparatus according to claim 14, wherein the characteristic distribution information absorption coefficient distribution information in the object.

17. The object information obtaining apparatus according to claim wherein the second distribution obtaining unit combines a plurality of pieces of absorption coefficient distribution information to obtain combined absorption coefficient distribution information.

18. The object information obtaining apparatus according to claim wherein the second distribution obtaining unit obtains concentration distribution information by using a plurality of pieces of combined absorption coefficient distribution information obtained by irradiating light of mutually different wavelengths between the plurality of pieces of combined absorption coefficient distribution information.

19. The object information obtaining apparatus according to claim 16, wherein the second distribution obtaining unit obtains concentration distribution information using a plurality pieces of absorption coefficient distribution information obtained by irradiating light of mutually different wavelength between the plurality of pieces of absorption coefficient distribution information.

20. The object information obtaining apparatus according to claim 19, wherein the concentration distribution information is oxygen saturation distribution information.
